(19) United States
(12) Patent Application Publication
(10) Pub. No.: US 2020/0085632 A1
(43) Pub. Date: Mar. 19, 2020

(54) ABSORBENT NEGATIVE PRESSURE DRESSING

(71) Applicant: KCI LICENSING, INC., San Antonio, TX (US)

(72) Inventors: Christopher B. Locke, Bournemouth (GB); Timothy M. Robinson, Blandford Forum (GB)

(21) Appl. No.: 16/571,839

(22) Filed: Sep. 16, 2019

Related U.S. Application Data
(60) Provisional application No. 62/732,285, filed on Sep. 17, 2018.

(51) Int. Cl. A61F 13/02; A61M 1/00

(52) U.S. Cl. CPC A61F 13/0216; A61F 13/0209; A61F 13/0206; A61M 2205/7527; A61M 1/009; A61M 2207/00; A61M 1/0086

(57) ABSTRACT

A dressing includes a drape sealable over a wound bed, a hydrophilic foam layer coupled to the drape, a plurality of superabsorbent dots positioned between the drape and the hydrophilic foam layer, a manifold layer positioned along the hydrophilic foam layer, and a plurality of superabsorbent dots positioned between the drape and the hydrophilic foam layer. The manifold layer is substantially pneumatically isolated from the superabsorbent dots by the hydrophilic foam layer. The dressing also includes one or more channels extending through the hydrophilic foam layer and a connection pad aligned with the one or more channels. The one or more channels provide pneumatic communication between the manifold layer and the connection pad. The connection pad is coupleable to a pump operable to create a negative pressure at the manifold layer.

### CROSS-REFERENCE TO RELATED APPLICATIONS

This application claims the benefit of priority to U.S. Provisional Application No. 62/732,285, filed on Sep. 17, 2018, which is incorporated herein by reference in its entirety.

### BACKGROUND

The present disclosure relates generally to the field of wound therapy, and more particularly to dressings for use in negative pressure wound therapy.

Negative pressure wound therapy (NPWT) is a type of wound therapy that involves applying negative pressure (relative to atmospheric pressure) to a wound bed to promote wound healing. Typically, a dressing is sealed over a wound bed and air is pumped out of the dressing to create a negative pressure at the wound bed. In some NPWT systems, wound exudate and other fluid is pumped out of the dressing and collected by a therapy system.

In other NPWT systems, air is pumped out of the dressing while the dressing is used to absorb fluid from the wound. In NPWT systems of this type, absorbent material of the dressing is typically subject to the negative pressure maintained by the pump. The negative pressure creates a squeezing force on the dressing that restricts expansion of the absorbent and limits the amount of fluid that the dressing can absorb. This may lead to reduced fluid absorption, the need for frequent dressing changes, or other challenges. A need therefore exists for a NPWT absorbent dressing that allows negative pressure to be maintained at a wound bed while enhancing the ability of the dressing to absorb fluid from the wound bed.

### SUMMARY

One implementation of the present disclosure is a dressing. The dressing includes a hydrophilic foam layer that includes a wound-facing side and a non-wound-facing side, a drape sealable over a wound bed, said drape positioned above the non-wound-facing side of the hydrophilic foam layer, a plurality of superabsorbent dots positioned between the drape and the hydrophilic foam layer, a manifold layer positioned under the wound-facing side of the hydrophilic foam layer. The manifold layer includes a wound-facing side and a non-wound facing side. The dressing also includes one or more channels extending through the hydrophilic foam layer and a connection pad in fluid communication with the one or more channels. The one or more channels provide fluid communication between the manifold layer and the connection pad. The connection pad is coupleable to a pump operable to create a negative pressure at the manifold layer.

In some embodiments, the dressing also includes a perforated film layer positioned under the wound-facing side of the manifold layer and allowing fluid to flow from the wound bed to the manifold layer. In some embodiments, the hydrophilic foam layer is configured to absorb fluid from the manifold layer and the superabsorbent dots are configured to absorb fluid from the hydrophilic foam layer.

In some embodiments, a portion of the drape covering the superabsorbent dots is free of adhesive. In some embodiments, the plurality of superabsorbent dots is separated from one another to facilitate deformation of the dressing.

In some embodiments, the dressing also includes a first fiber layer that binds the drape to the hydrophilic foam layer and secures the superabsorbent dots to the hydrophilic foam layer. The drape may also include a second fiber layer that binds the hydrophilic foam layer to the manifold layer. In some embodiments, the drape also includes a hydrophobic filter positioned between the one or more channels and the connection pad.

Another implementation of the present disclosure is a negative pressure wound therapy system. The negative pressure wound therapy system includes a pump operable to create a negative pressure, a tube coupled to the pump, and a dressing coupled to the tube. The dressing includes a drape sealable over a wound bed, a hydrophilic foam layer coupled to the drape, a plurality of superabsorbent dots positioned between the drape and the hydrophilic foam layer, and a manifold layer positioned under the hydrophilic foam layer. The manifold layer is substantially pneumatically isolated from the superabsorbent dots by the hydrophilic foam layer. The dressing also includes one or more channels extending through the hydrophilic foam layer and a connection pad aligned with the one or more channels. The one or more channels provide fluid communication between the manifold layer and the connection pad, and the connection pad is coupleable to the tube to provide fluid communication between the pump and the manifold layer.

In some embodiments, the pump is manually powered. In some embodiments, the dressing also includes a perforated film layer positioned along the manifold layer and allows fluid to flow from the wound bed to the manifold layer.

In some embodiments, the hydrophilic foam layer is configured to absorb fluid from the manifold layer and the superabsorbent dots are configured to absorb fluid from the hydrophilic foam. In some embodiments, the drape includes a porous material that allows evaporation of fluid absorbed by the superabsorbent dots through the drape.

In some embodiments, the dressing includes a first fiber layer that binds the drape to the hydrophilic foam layer and secures the superabsorbent dots to the hydrophilic foam layer. The drape may also include a second fiber layer that binds the hydrophilic foam layer to the manifold layer.

In some embodiments, the dressing also includes a hydrophobic filter positioned between the one or more channels and the connection pad. In some embodiments, the superabsorbent dots are maintained at substantially atmospheric pressure when the pump creates a negative pressure at the manifold layer.

Another implementation of the present disclosure is a method of manufacturing a dressing. The method includes printing a plurality of superabsorbent dots on a hydrophilic foam layer, creating one or more channels through the hydrophilic foam layer, and coupling the hydrophilic foam layer to a drape. The superabsorbent dots are positioned between the hydrophilic foam layer and the drape. The method also includes coupling a manifold layer to the hydrophilic foam layer in fluid communication with the one or more channels. The manifold layer is substantially pneumatically isolated from the superabsorbent dots by the hydrophilic foam layer. The method further includes coupling a connection pad to the drape in fluid communication with the one or more channels. The connection pad is coupleable to a pump operable to create a negative pressure at the manifold layer.

In some embodiments, printing the plurality of superabsorbent dots on the hydrophilic foam layer comprises includes a superabsorbent polymer in a pattern on the hydrophilic foam layer. The pattern may include unconnected dots.

In some embodiments, the method also includes coupling a perforated film layer to the manifold layer. The perforated film layer is coupleable to a wound bed and configured to allow fluid to flow from the wound bed to the manifold layer.

In some embodiments, the hydrophilic foam layer is configured to absorb fluid from the manifold layer and the superabsorbent dots are configured to absorb fluid from the hydrophilic foam layer. In some embodiments, the drape includes a porous material that allows evaporation of fluid absorbed by the superabsorbent dots through the drape.

In some embodiments, coupling the hydrophilic foam layer to the drape includes binding the hydrophilic foam layer to the drape with a fusible fiber layer positioned between the drape and the hydrophilic foam layer. The fusible fiber layer secures the superabsorbent dots to the hydrophilic foam layer. In some embodiments, coupling the manifold layer to the hydrophilic foam layer includes fusing a fusible fiber layer between the hydrophilic foam layer and the manifold layer.

In some embodiments, the method also includes positioning a hydrophobic filter between the one or more channels and the connection pad.

### BRIEF DESCRIPTION OF THE DRAWINGS

FIG. 1 is a block diagram of a negative pressure wound therapy (NPWT) system, according to an exemplary embodiment.

FIG. 2 is an exploded perspective view of a dressing for use with the NPWT system of FIG. 1, according to an exemplary embodiment.

FIG. 3 is a schematic cross-sectional side view of the dressing of FIG. 2, according to an exemplary embodiment.

FIG. 4 is a top view of a first embodiment of a portion of the dressing of FIG. 2, according to an exemplary embodiment.

FIG. 5 is a top view of a second embodiment of a portion of the dressing of FIG. 2, according to an exemplary embodiment.

FIG. 6 is a top view of a third embodiment of a portion of the dressing of FIG. 2, according to an exemplary embodiment.

FIG. 7 is a top view of a fourth embodiment of a portion of the dressing of FIG. 2, according to an exemplary embodiment.

FIG. 8 is a graph of experimental results from a first experiment using the NPWT system of FIG. 1, according to an exemplary embodiment.

FIG. 9 is a graph of experimental results from a second experiment using the NPWT system of FIG. 1, according to an exemplary embodiment.

### DETAILED DESCRIPTION

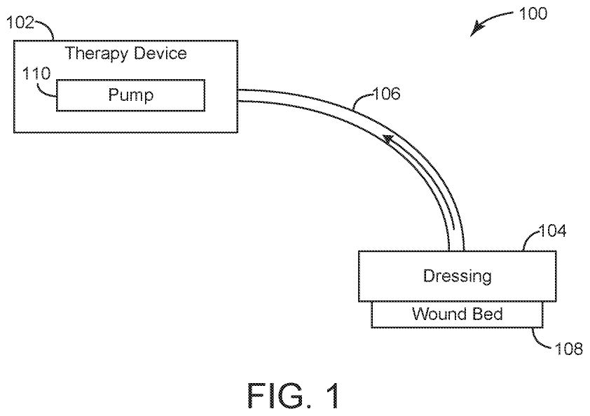

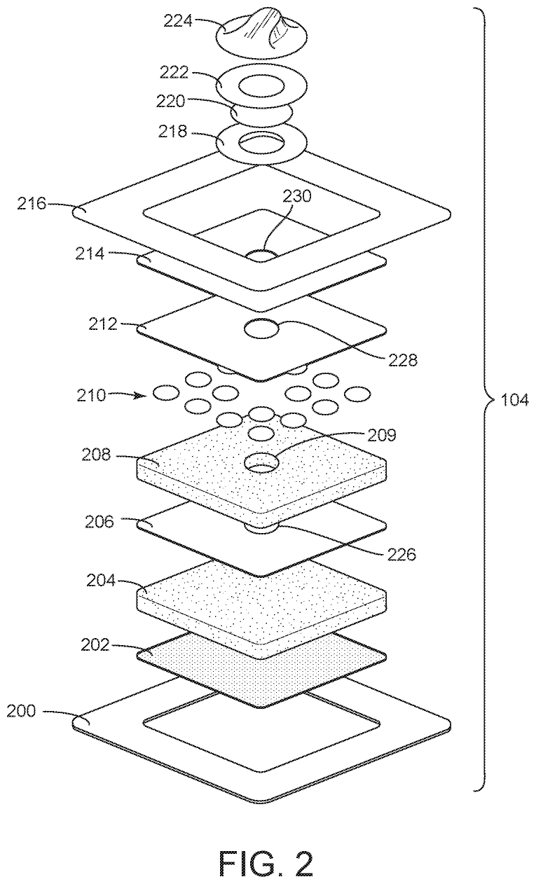

Referring now to FIG. 1, a negative pressure wound therapy (NPWT) system 100 is shown, according to an exemplary embodiment. The NPWT system 100 includes a therapy device 102 pneumatically communicable with a dressing 104 via tube 106. The dressing 104 is shown as sealed over a wound bed 108. The wound bed 108 is a tissue wound of a patient, for example a laceration, burn, sore, trauma wound, chronic wound, etc. As shown in FIGS. 2-4 and described in detail with reference thereto, the dressing 104 allows a negative pressure to be maintained at the wound bed 108 while absorbing fluid from the wound bed 108 with superabsorbent dots pneumatically isolated from the negative pressure. The dressing 104 thereby provides both negative pressure and a high level of fluid absorption not found in conventional NPWT dressings.

The therapy device 102 includes a pump 110. The pump 110 is operable to pump air out of the dressing 104 via the tube 106 to create and maintain a negative pressure at the wound bed 108. In some embodiments, the pump 110 is electrically powered and the therapy device 102 includes power systems and control circuitry to power and control operation of the pump 110. For example, the therapy device 102 may include one or more pressure sensors or various other sensors that collect data used by the therapy device 102 in controlling the pump 110 to maintain a negative pressure at the wound bed 108. In some embodiments, the pump 110 is manually-powered, such that a user may manipulate the pump 110 to draw air out of the dressing 104 as desired by the user. For example, the pump 110 may be spring-loaded to gradually pull air from the dressing 104 for a duration of time following a compression of the pump 110 by the user.

In some embodiments, the therapy device 102 includes a control circuit configured to detect when the dressing 104 is full, i.e., when the dressing 104 has absorbed a threshold amount of fluid. For example, the control circuit may use a dead-space detection approach in which pressure is released and a pressure decay time is measured. When the dressing 104 is full, there is little or no open volume at the dressing 104, decreasing the decay time. The control circuit may determine that the decay time is less than a threshold decay time and, in response, generate an alert for a user informing the user that the dressing 104 is full.

The NPWT system 100 is thereby configured to provide a negative pressure at the wound bed 108 while also facilitating absorption of fluid from the wound bed 108 by the dressing 104.

Referring now to FIG. 2, an exploded perspective view of the dressing 104 is shown, according to an exemplary embodiment. The dressing 104 includes a plurality of layers, including a sealing adhesive layer 200 sealable around the wound bed 108, a perforated film layer 202 configured to abut the wound bed 108, a manifold layer 204 positioned along the perforated film layer 202, a first fusible fiber layer 206 that binds the manifold layer 204 to a hydrophilic foam layer 208, a plurality of superabsorbent dots 210 distributed on the hydrophilic foam layer 208, a second fusible fiber layer 212 that binds the hydrophilic foam layer 208 to a non-adhesive drape 214, an adhesive drape 216 sealable around the non-adhesive drape 214, a filter 220 coupled to the non-adhesive drape 214 by a first adhesive ring 218, and a connection pad 224 aligned with the filter 220 and coupled to the filter 220 by a second adhesive ring 222.

The sealing adhesive layer 200 forms a border of the dressing 104. The sealing adhesive layer 200 is sealable to a patient's skin surrounding the wound bed 108 to secure the dressing 104 to the patient. The sealing adhesive layer 200 substantially prevents air from leaking between the dressing 104 and the patient's skin to facilitate creation of a negative pressure at the wound bed 108. The sealing adhesive layer 200 may include one or more adhesives, for example a combination of an acrylic adhesive and a silicone gel that provides a secure seal while also facilitating substantially painless and harmless removal of the dressing 104 from the wound bed 108. As one illustrative example, the sealing adhesive layer 200 may include DERMATAC™ by Acelity™. As another illustrative example, the sealing adhesive layer 200 may include a tri-laminate adhesive silicone gel commercially available from Scapa Healthcare and marketed as Scapa Soft-Pro Silicone Gel 6058.

The perforated film layer 202 is positioned within the border formed by the sealing adhesive layer 200. The perforated film layer 202 is configured to provide a gentle, low-tack interface between the dressing 104 and the wound bed 108, for example to facilitate removal of the dressing 104 from the wound bed 108 without substantial disruption to the healing process. The perforated film layer 202 includes a plurality of perforations that allows wound exudate to pass therethrough and allows a negative pressure in the manifold layer 204 to reach the wound bed 108. For example, the perforated film layer 202 may include a plurality of slits having dimensions of approximately two millimeters to three millimeters by one-half millimeter. The perforated film layer 202 may be manufactured from polyurethane or some other suitable material. As one illustrative example, the perforated film layer 202 may include a material commercially available from Coveris and marketed as Inspire 2327.

The manifold layer 204 is positioned along the perforated film layer 202. The manifold layer 204 is configured to allow air to flow therethrough, facilitating the distribution of negative pressure across the wound bed 108. The manifold layer 204 is also structured to allow the flow of wound exudate from the wound bed 108 to the hydrophilic foam layer 208. The manifold layer 204 is made of a hydrophobic open-cell foam, one illustrative example of which is GRANUFOAM™ by Acelity™. In other embodiments, the manifold layer 204 may be made of a manifolding three-dimensional fabric, examples of which may be commercially available from Baltex. In some embodiments, the manifold layer 204 has a thickness between two millimeters and eight millimeters.

The first fusible fiber layer 206 binds the manifold layer 204 to the hydrophilic foam layer 208, aligning the hydrophilic foam layer 208 with the manifold layer 204 and the perforated film layer 202. The first fusible fiber layer 206 is fused to both the manifold layer 204 and the hydrophilic foam layer 208. The first fusible fiber layer 206 has an open, flexible structure that allows the flow of fluid therethrough and does not limit the flexibility or conformability of the dressing 104. As one illustrative example, the first fusible fiber layer 206 may include a material commercially available from Freudenberg and marketed under the designation M1590. Other suitable materials may also be used. The first fusible fiber layer 206 may include a hole 226 that allows unimpeded airflow through the first fusible fiber layer 206 (i.e., through the hole 226). In alternative embodiments, the first fusible fiber layer 206 may be omitted, for example in an embodiment where the hydrophilic foam layer 208 is configured to be directly fused to the manifold layer 204.

The hydrophilic foam layer 208 absorbs fluid from the wound bed 108 via the perforated film layer 202 and the manifold layer 204. The hydrophilic foam layer 208 is made of a substantially-closed-cell hydrophilic foam. For example, the hydrophilic foam layer 208 may be made of aromatic or aliphatic polyurethanes. The hydrophilic foam layer 208 is substantially impermeable to air, substantially preventing the flow of air therethrough. In some embodiments, the hydrophilic foam layer 208 allows some airflow therethrough when dry and becomes more impermeable to air as the hydrophilic foam layer 208 absorbs fluid. In some embodiments, the hydrophilic foam layer 208 may include a polyvinyl alcohol dressing such as WHITEFOAM™ by Acelity™.

The hydrophilic foam layer 208 thereby substantially isolates the superabsorbent dots 210 from a negative pressure at the manifold layer 204. That is, the hydrophilic foam layer 208 is configured to preserve a pressure differential across the hydrophilic foam layer 208, for example allowing the superabsorbent dots 210 to experience atmospheric pressure while the manifold layer 204 is at a negative pressure relative to atmospheric pressure. In alternative embodiments, the hydrophilic foam layer 208 may be replaced by a film layer that is configured to allow fluid to pass through the film layer while preventing the transmission of air pressure across the film layer.

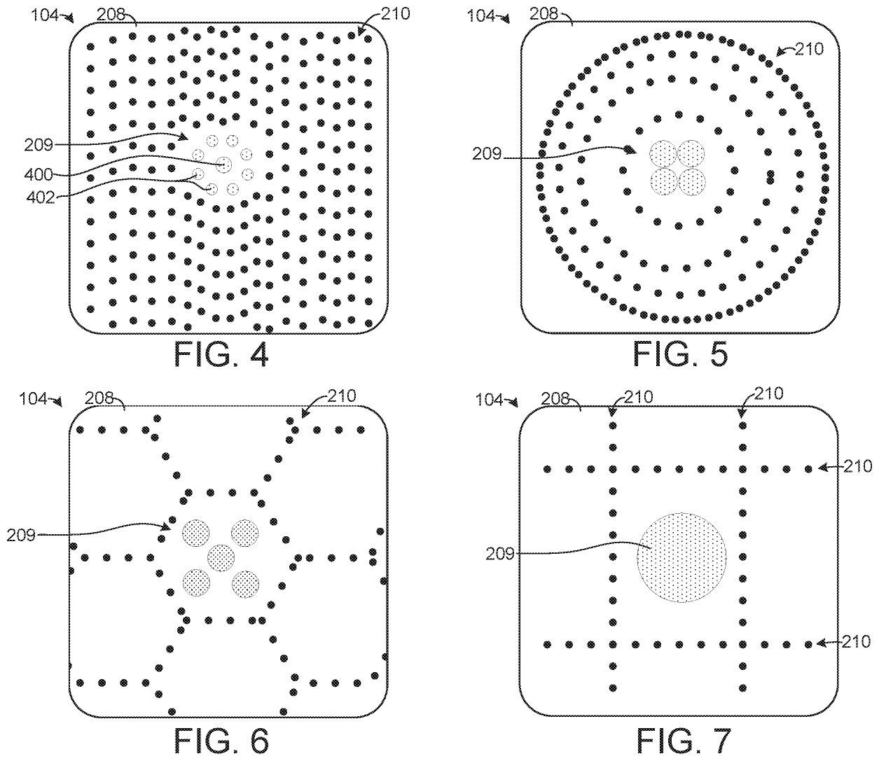

One or more channels 209 extend through the hydrophilic foam layer 208 and allow air to flow from the manifold layer 204 to the connection pad 224. In some embodiments, one channel 209 extends through the hydrophilic foam layer 208, for example as shown in FIG. 2. The channel 209 may have a diameter between ten and twenty millimeters. In other embodiments, multiple channels 209 extend through the hydrophilic foam layer 208, for example as shown in FIG. 4 and described in detail with reference thereto. In such embodiments, each channel 209 may have a diameter between two and three millimeters, with the multiple channels 209 positioned proximate one another, for example within an area with a diameter of twenty millimeters. The one or more channels 209 may each have a circular shape, square shape, rectangular shape, elliptical shape, or some other shape.

The superabsorbent dots 210 are positioned on the hydrophilic foam layer 208, with the hydrophilic foam layer 208 between the superabsorbent dots 210 and the manifold layer 204. The superabsorbent dots 210 are made of one or more materials that absorb a large amount of fluid (e.g., sodium polyacrylate, polyacrylamide copolymer, ethylene maleic anhydride copolymer, cross-linked carboxymethylcellulose, polyvinyl alcohol copolymers, cross-linked polyethylene oxide). As one illustrative example, in some embodiments the superabsorbent dots 210 include a material commercially available from Bayer and marketed as Luquasorb 1161.

Each superabsorbent dot 210 may be configured to absorb up to thirty to sixty times the volume of the superabsorbent dot 210 in water or other fluid. The superabsorbent dots 210 are highly hydrophilic, wicking fluid from the hydrophilic foam layer 208. According to various embodiments, the superabsorbent dots 210 are arranged on the hydrophilic foam layer 208 in various patterns, densities, distributions, etc. In some embodiments, the superabsorbent dots 210 are separated from one another to facilitate deformation of the dressing 104. Example arrangements of the superabsorbent dots 210 are illustrated in FIGS. 4-7.

The second fusible fiber layer 212 is fused to the hydrophilic foam layer 208 and secures the superabsorbent dots 210 to the hydrophilic foam layer 208. The second fusible fiber layer 212 also binds the hydrophilic foam layer 208 to the non-adhesive drape 214.

The second fusible fiber layer 212 has an open, flexible structure that allows the flow or evaporation of fluid therethrough and does not limit the flexibility or conformability of the dressing 104. The second fusible fiber layer 212 may include a hole 228 aligned with the one or more channels 209 that allows unimpeded airflow through the second fusible fiber layer 212 (i.e., through the hole 228). In alternative embodiments, the second fusible fiber layer 212 may be omitted, for example in an embodiment where the hydrophilic foam layer 208 is configured to be directly fused to non-adhesive drape 214.

The non-adhesive drape 214 is positioned along the hydrophilic foam layer 208 and is configured to allow evaporation of fluid from the hydrophilic foam layer 208 and the superabsorbent dots 210 through the non-adhesive drape 214 to the environment. In some embodiments, the non-adhesive drape 214 directly contacts the superabsorbent dots 210. The non-adhesive drape 214 may be flexible and/or stretchable to maintain contact with or close proximity to the superabsorbent dots 210 while the superabsorbent dots 210 expand to absorb fluid and contract as fluid evaporates. The non-adhesive drape 214 may be between twenty and fifty microns in thickness. As an illustrative example, in some embodiments the non-adhesive drape 214 includes the same material or materials as the V.A.C.® drape by ACELITY™. The non-adhesive drape 214 includes a hole 230 aligned with the one or more channels 209 and the holes 226, 228 to allow airflow between the manifold layer 204 and the connection pad 224.

The adhesive drape 216 surrounds the non-adhesive drape 214 and covers the periphery of the dressing 104. The adhesive drape 216 may be made of an identical or similar material as the non-adhesive drape 214, further including an adhesive on an underside of the adhesive drape 216. The adhesive drape 216 forms a ring around the non-adhesive drape 214, overlapping with the non-adhesive drape 214 peripherally by between five and eight millimeters to allow the adhesive drape 216 to bind to the non-adhesive drape 214. The adhesive drape 216 may also bind to the sealing adhesive layer 200, enclosing the manifold layer 204, the first fusible fiber layer 206, the hydrophilic foam layer 208, the superabsorbent dots 210, and the second fusible fiber layer 212 in a volume defined by the sealing adhesive layer 200, the perforated film layer 202, the adhesive drape 216, and the non-adhesive drape 214.

A filter 220 is aligned with the one or more channels 209 and coupled to the non-adhesive drape 214 by the first adhesive ring 218. The filter 220 includes a hydrophobic filter material that is impermeable to fluids (i.e., liquids) but permeable to air. Accordingly, the filter 220 allows air to flow therethrough from the one or more channels 209 (i.e., from the manifold layer 204) to the connection pad 224, while preventing fluid from entering the connection pad 224. As one illustrative example, in some embodiments the filter 220 includes a material commercially available from Gore and designated as MMT314. In some embodiments, the filter 220 also includes a charcoal filter material structured to reduce odors released via the filter 220, for example a material available from Calgon Carbon and marketed as Zorflex.

The first adhesive ring 218 has an outside diameter slightly larger than a diameter of the filter 220 and an inside diameter equal or close to a diameter of the hole 230 in the non-adhesive drape 214 aligned with the one or more channels 209. For example, in some embodiments, the first adhesive ring 218 and an inside diameter of approximately twenty-six millimeters and an outside diameter of approximately forty-one millimeters, while the filter 220 has a diameter of approximately thirty-two millimeters. In such embodiments, the holes 230, 228, and 226 may also have a diameter of approximately twenty-six millimeters. The first adhesive ring 218 includes a double-sided adhesive that binds the filter 220 to the non-adhesive drape 214. As one illustrative example, in some embodiments the first adhesive ring 218 includes a material commercially available from Lohmann and designated as Duplocoll 20606. A second adhesive ring 222 is aligned with the first adhesive ring 218 and positioned to sandwich the filter 220 between the first adhesive ring 218 and the second adhesive ring 222. The second adhesive ring 222 may be substantially the same as the first adhesive ring 218.

The connection pad 224 is aligned with the filter 220 and the one or more channels 209 and is coupled to the non-adhesive drape 214 by the second adhesive ring 222. The connection pad 224 is coupleable to the tube 106 shown in FIG. 1 to place the one or more channels 209 in pneumatic communication with the tube 106 and the pump 110 via the filter 220 and the connection pad 224. The connection pad 224 thereby facilitates connection between the dressing 104 and the therapy device 102. The connection pad 224 may be manufactured from injection-molded polyurethane.

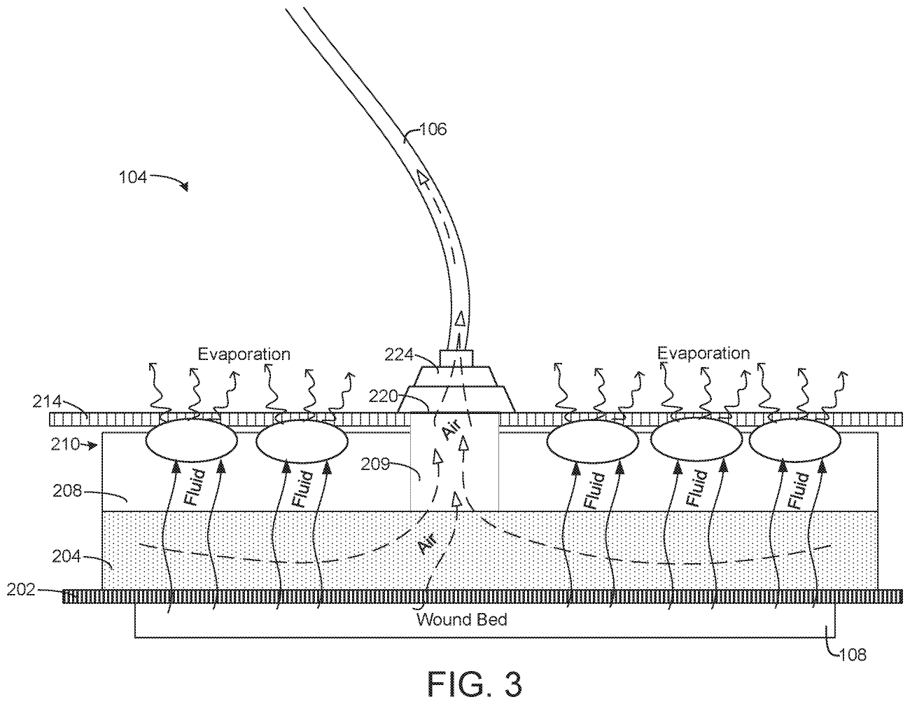

Referring now to FIG. 3, a schematic cross-sectional side view of the dressing 104 is shown, according to an exemplary embodiment. The schematic cross-sectional side view of FIG. 3 illustrates the flow of air and fluid through the dressing 104.

As shown in FIG. 3, the perforated film layer 202 is positioned abutting the wound bed 108, the manifold layer 204 abuts the perforated film layer 202, and the hydrophilic foam layer 208 is positioned along the manifold layer 204. The superabsorbent dots 210 are positioned on the hydrophilic foam layer 208, with the hydrophilic foam layer 208 separating the superabsorbent dots 210 from the manifold layer 204. The non-adhesive drape 214 is positioned along the hydrophilic foam layer 208, for example in contact with the superabsorbent dots 210. The filter 220 and the connection pad 224 are coupled to the non-adhesive drape 214 and positioned over the one or more channels 209 (depicted as a single channel 209 in FIG. 3) through the hydrophilic foam layer 208.

As illustrated by the solid-line arrows in FIG. 3, the dressing 104 is configured to wick fluid (e.g., wound exudate) from the wound bed 108, absorb the fluid, and allow the fluid to evaporate to the environment. More particularly, the perforated film layer 202 and the manifold layer 204 facilitate the flow of fluid from the wound bed 108 to the hydrophilic foam layer 208. The hydrophilic foam layer 208 is hydrophilic and accordingly attracts water-based fluids, which includes most wound exudate fluids. The hydrophilic foam layer 208 thereby receives and absorbs fluid. The superabsorbent dots 210 receive and absorb fluid from the hydrophilic foam layer 208. The superabsorbent dots 210 may be more hydrophilic than the hydrophilic foam layer 208, drawing fluid from the hydrophilic foam layer 208 up to the superabsorbent dots 210. Thus, the relative positioning of the manifold layer 204, the hydrophilic foam layer 208, and the superabsorbent dots 210 provides a moisture flow gradient that draws fluid away from the wound bed 108.

As shown in FIG. 3, the superabsorbent dots 210 are positioned proximate the non-adhesive drape 214, which allows fluid to evaporate from the superabsorbent dots 210 via the non-adhesive drape 214. Because the superabsorbent dots 210 expand when the superabsorbent dots 210 absorb fluid, the non-adhesive drape 214 may be configured to stretch or flex to maintain proximity between the non-adhesive drape 214 and the superabsorbent dots 210 as the superabsorbent dots 210 change size. The dressing 104 may thereby facilitate evaporation through the non-adhesive drape 214 at any fill level of the dressing 104. Furthermore, the swelling of each of the superabsorbent dots 210 may be observable by a patient or caregiver, indicating to the patient or caregiver which regions of the wound bed 108 are providing the most fluid (e.g., exuding the most wound exudate). The superabsorbent dots 210 may thereby provide information about the behavior of the wound bed 108 that may be used to modify or customize treatment of the wound bed 108.

As illustrated by the dashed-line arrows in FIG. 3, the dressing 104 also facilitates the flow of air through the dressing 104 to allow the therapy device 102 to draw a negative pressure in the manifold layer 204 and at the wound bed 108. The perforated film layer 202 allows air to flow therethrough (e.g., through perforations in the perforated film layer 202) from the wound bed 108 to the manifold layer 204 and vice versa. The wound bed 108 is thereby exposed to the air pressure of the manifold layer 204 (e.g., a negative pressure). As mentioned above, the manifold layer 204 includes an open-celled foam that allows air to flow freely throughout the manifold layer 204, ensuring that the air pressure of the manifold layer 204 is substantially uniformly distributed throughout the manifold layer 204.

The one or more channels 209 are in pneumatic communication with the manifold layer 204, such that air can flow between the one or more channels 209 and the manifold layer 204. The filter 220 and the connection pad 224 are aligned with the one or more channels 209, such that air may flow from the one or more channels 209, through the filter 220, and into the connection pad 224. The tube 106 is coupled to the connection pad 224 to allow air to flow from the connection pad 224 through the tube 106 to the pump 110 shown in FIG. 1.

To create a negative pressure at the wound bed 108, the pump 110 operates to pump air out of the tube 106, thereby pulling air from the manifold layer 204, through the one or more channels 209, through the filter 220, and through the connection pad 224 to the tube 106. By removing air from the manifold layer 204, the pump 110 creates a negative pressure relative to atmospheric pressure in the manifold layer 204 and at the wound bed 108. The filter 220 is hydrophobic and prevents fluid or other debris from entering the connection pad 224 and the tube 106 while allowing air to pass from the one or more channels 209 into the tube 106.

As illustrated by FIG. 3, the hydrophilic foam layer 208 substantially prevents the flow of air therethrough. The hydrophilic foam layer 208 thereby substantially isolates the superabsorbent dots 210 from a negative pressure of the manifold layer 204, allowing the superabsorbent dots 210 to experience substantially atmospheric pressure while the manifold layer 204 is at a negative pressure relative to atmospheric pressure. For example, at a therapy pressure (i.e., negative pressure of the manifold layer 204) of approximately 125 mmHG, the superabsorbent dots 210 may experience a pressure of less than approximately 5 mmHG. Any residual pressure at the level of the superabsorbent dots 210 may act to ensure close contact between the superabsorbent dots 210 and the non-adhesive drape 214.

The negative pressure of the manifold layer 204 may create a force on the hydrophilic foam layer 208 directed towards the manifold layer 204 (i.e., forcing the hydrophilic foam layer 208 towards the manifold layer 204) because of the pressure differential across the hydrophilic foam layer 208. However, the superabsorbent dots 210 are substantially isolated from the negative pressure by the hydrophilic foam layer 208 and experience little or no such restrictive or compressive force. Thus, the superabsorbent dots 210 are free to absorb fluid and greatly expand without restriction caused by the negative pressure. The hydrophilic foam layer 208 and the superabsorbent dots 210 may absorb fluid with or without the negative pressure applied by the pump 110, thereby providing fail-safe reliability for the dressing 104.

The dressing 104 thereby provides for the application of negative pressure wound therapy to the wound bed 108 while also providing unencumbered absorption of wound exudate.

Referring now to FIGS. 4-7, top views of various embodiments of the hydrophilic foam layer 208 with superabsorbent dots 210 are shown, according to exemplary embodiments. FIGS. 4-7 illustrate various distributions, densities, shapes, arrangements etc. of the superabsorbent dots 210 on the hydrophilic foam layer 208. FIGS. 4-7 also illustrate various configurations of the one or more channels 209. It should be understood that the embodiments of FIGS. 4-7 are included for illustrative purposes and that various other distributions, densities, arrangements etc. of the superabsorbent dots 210 and configurations of the one or more channels 209 are contemplated by the present disclosure. For example, different distributions, densities, and arrangements of the superabsorbent dots 210 may be chosen to vary the absorption capacity of the dressing 104 (i.e., the amount fluid that the dressing 104 can absorb) or to customize the moisture flow gradient (e.g., to draw fluid towards the perimeter of the dressing 104, towards the center of the dressing 104, towards one side of the dressing 104, etc.)

In the embodiment of FIG. 4, the superabsorbent dots 210 are approximately evenly distributed over the hydrophilic foam layer 208. The superabsorbent dots 210 thereby provide fluid absorption across all regions of the wound bed 108. The channels 209 include a central channel 400 and multiple (shown as eight) secondary channels 402 surrounding the central channel 400. The central channel 400 may be larger than the secondary channels 402. For example, the central channel 400 may have a diameter of approximately three millimeters while the secondary channels 402 have a diameter of approximately two millimeters. The central channel 400 and the multiple secondary channels 402 are grouped to fit within an area coverable by the filter 220 and the connection pad 224.

In the embodiment of FIG. 5, the superabsorbent dots 210 are arranged in concentric rings around the channels 209. A higher density of superabsorbent dots 210 is found at the outer, larger rings, for example to customize the moisture flow gradient of the dressing 104 to draw fluid towards the periphery of the dressing 104. The hydrophilic foam layer 208 includes four circular (i.e., cylindrical) channels 209 in a square arrangement extending through the hydrophilic foam layer 208. In the embodiment of FIG. 5, the superabsorbent dots 210 are arranged in tessellated hexagons on the hydrophilic foam layer 208. The channels 209 include five channels 209 arranged in a cross shape (i.e., as on the “5” side of gaming dice). In the embodiment of FIG. 7, the superabsorbent dots 210 are arranged in four lines (stripes, rows) and a single channel 209 extends through the hydrophilic foam layer 208. The channel 209 may have a diameter between approximately fifteen millimeters and twenty millimeters.

As illustrated by the various embodiments of FIGS. 4-7, the dressing 104 may include various distributions, densities, arrangements etc. of the superabsorbent dots 210 and various configurations of the one or more channels 209. In the embodiments shown, the superabsorbent dots 210 are separated from one another to facilitate deformation of the dressing 104 to allow the dressing 104 to conform to the geometry of the wound bed 108 and to avoid limiting the expansion of each superabsorbent dot 210. In alternative embodiments, the superabsorbent dots 210 are joined in lines, stripes, solid shapes, or other geometric forms. In some embodiments, the superabsorbent dots 210 form a layer of a superabsorbent material that substantially covers the hydrophilic foam layer 208. In some embodiments, the superabsorbent dots 210 are round (e.g., circular, elliptical). In various other embodiments, the superabsorbent dots 210 may be formed as other shapes (rectangles, squares, triangles, chevrons, pentagons, etc.), lines, irregular forms, etc. Accordingly the term “dots” is intended to have broad meaning and the superabsorbent dots

The superabsorbent dots 210 may be printed on the hydrophilic foam layer 208. For example, in some embodiments a superabsorbent polymer is formed into a solution, dripped onto the hydrophilic foam layer 208 in a desired pattern or arrangement, and then cross-linked (e.g., with ultraviolet light) to form a gel (i.e., hydrogel). In other embodiments, super-absorbent polymer granules are dispersed to form a slurry in a water sensitive carrier (e.g., polyvinyl alcohol, polyvinyl pyrrolidone, or polyvinyl acetate), and printed (e.g., dripped on the hydrophilic foam layer 208 in a desired pattern). Such methods of printing the superabsorbent dots 210 on the hydrophilic foam layer 208 may cause the superabsorbent dots 210 to bind to the hydrophilic foam layer 208 and prevent loss of superabsorbent dots 210 when the dressing 104 is cut or otherwise modified or manipulated.

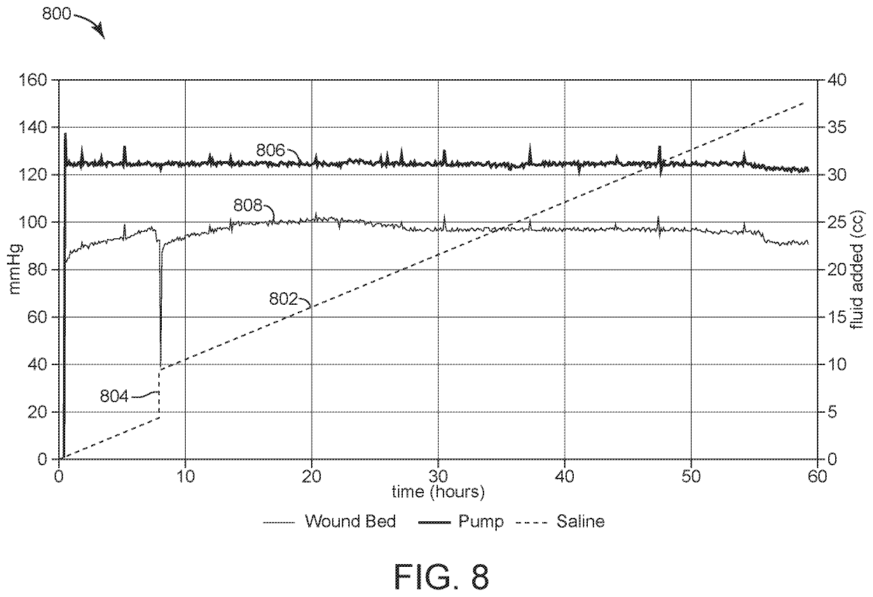

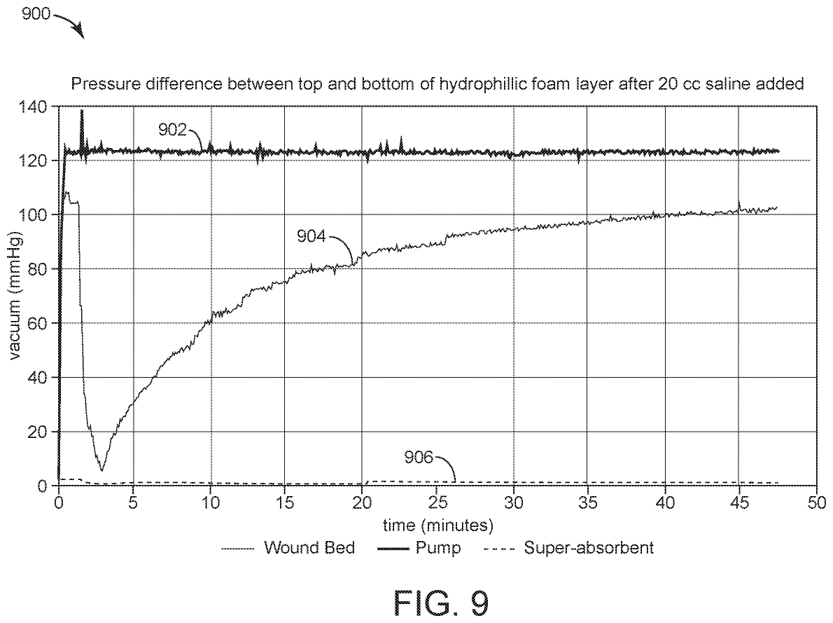

Referring now to FIGS. 8-9, experimental results of experiments that use the NPWT system 100 are shown, according to an exemplary embodiment. FIGS. 8-9 correspond to two different experiments. In each experiment, the pump 110 is operated to pull air from the manifold layer 204 and saline solution is added to the dressing 104. Measurements of air pressure at various locations of the NPWT system 100 are taken and shown on the graphs of FIGS. 8-10. It should be understood that while FIGS. 8-10 are included to illustrate the advantages and properties of one embodiment of the dressing 104, results may vary across various implementations of the present disclosure.

FIG. 8 shows a graph 800 that depicts the results of an experiment in which saline solution is added to the dressing 104 at approximately 0.56 cubic centimeters per hour over two and a half days. The graph 800 charts pressure and fluid added over a time period of sixty hours. The saline line 802 represents the total amount of saline solution added to the dressing 104 over time. The saline line 802 increases linearly, with the exception of a step 804 at a time of approximately eight hours. The pump line 806 represents the pressure at the pump 110 (e.g., in the tube 106). As shown in FIG. 8, the pump 110 operates at a pressure of approximately 125 mmHG relative to atmospheric pressure. Apart from some noise, the pump line 806 shows that the pressure at the pump 110 is substantially constant in the experiment of FIG. 8.

The wound bed line 808 represents the pressure at the wound bed 108 over time. The wound bed line 808 shows some lag time of the pressure at the wound bed 108 increasing to a stable pressure, both at the beginning of the experiment and after the step 804 in the amount of fluid added to the dressing 104. The pressure at the wound bed 108 (connected to the pump 110 via the manifold layer 204, the one or more channels 209, the filter 220, the connection pad 224, and the tube 106) is shown to reach a relatively stable pressure of approximately 100 mmHG. As illustrated by FIG. 8, the wound bed line 808 may trend downward slightly towards the end of the experiment, indicating a potential loss of negative pressure at the wound bed 108 as the dressing 104 fills with fluid.

FIG. 9 shows a graph 900 that depicts the results of an experiment in which twenty cubic centimeters of saline solution are added to the dressing 104 near the beginning of the experiment, which runs for less than fifty minutes (i.e., substantially shorter than the experiment of FIG. 10). The graph 900 includes a pump line 902 that shows that the pump 110 operates at a substantially constant pressure of 125 mmHG exerted on the tube 106 coupled to the dressing 104. The graph 900 also includes a wound bed line 904 that represents the pressure at the wound bed 108 (i.e., at the manifold layer 204). When the fluid is added, the wound bed line 904 indicates that the pressure at the wound bed 108 approaches atmospheric pressure. Over the remainder of the experiment, the wound bed line 904 slopes upwards, approaching approximately 100 mmHG. Thus, graph 900 illustrates that the NPWT system 100 allows a negative pressure of approximately 100 mmHG to be established at the wound bed 108.

The graph 900 also includes a superabsorbent line 906. The superabsorbent line 906 represents the pressure at the level of the superabsorbent dots 210, i.e., on the opposite side of the hydrophilic foam layer 208 as the manifold layer 204. The difference between the wound bed line 904 and the hydrophilic foam layer 208 represents the pressure differential across the hydrophilic foam layer 208. As shown in FIG. 9, the superabsorbent line 906 remains substantially at zero mmHG, i.e., at atmospheric pressure. The graph 900 illustrates that a pressure differential of up to at least 100 mmHG across the hydrophilic foam layer 208 may be established in the dressing 104. Thus, the superabsorbent dots 210 may be held at substantially atmospheric pressure while a significant negative pressure is created and maintained at the wound bed 108.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and are considered to be within the scope of the disclosure.

Other arrangements and combinations of the elements described herein and shown in the Figures are also contemplated by the present disclosure. The construction and arrangement of the systems and apparatuses as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. Other substitutions, modifications, changes, and omissions can be made in the design, operating conditions and arrangement of the exemplary embodiments without departing from the scope of the present disclosure.

What is claimed is:

1. A dressing, comprising:
a hydrophilic foam layer comprising a wound-facing side and a non-wound-facing side;
a drape sealable over a wound bed, said drape positioned above the non-wound-facing side of the hydrophilic foam layer
a plurality of superabsorbent dots positioned between the drape and the hydrophilic foam layer;
a manifold layer positioned under the wound-facing side of the hydrophilic foam layer the manifold layer comprising a wound-facing side and a non-wound-facing side;
one or more channels extending through the hydrophilic foam layer;
a connection pad in fluid communication with the one or more channels, the one or more channels providing fluid communication between the manifold layer and the connection pad;
wherein the connection pad is coupleable to a pump operable to create a negative pressure at the manifold layer.

2. The dressing of claim 1, further comprising a perforated film layer positioned under the wound-facing side of the manifold layer and allowing fluid to flow from the wound bed to the manifold layer.

3. The dressing of claim 1, wherein the hydrophilic foam layer is configured to absorb fluid from the manifold layer and the superabsorbent dots are configured to absorb fluid from the hydrophilic foam layer.

4. The dressing of claim 1, wherein a portion of the drape covering the superabsorbent dots is free of adhesive.

5. The dressing of claim 1, wherein the plurality of superabsorbent dots are separated from one another to facilitate deformation of the dressing.

6. The dressing of claim 1, further comprising:
a first fiber layer that binds the drape to the hydrophilic foam layer and secures the superabsorbent dots to the hydrophilic foam layer; and
a second fiber layer that binds the hydrophilic foam layer to the manifold layer.

7. The dressing of claim 1, further comprising a hydrophobic filter positioned between the one or more channels and the connection pad.

8. A negative pressure wound therapy system, comprising:
a pump operable to create a negative pressure;
a tube coupled to the pump;
a dressing coupled to the tube, the dressing comprising:
a drape sealable over a wound bed;
a hydrophilic foam layer coupled to the drape;
a plurality of superabsorbent dots positioned between the drape and the hydrophilic foam layer;
a manifold layer positioned under the hydrophilic foam layer, the manifold layer substantially pneumatically isolated from the superabsorbent dots by the hydrophilic foam layer;
one or more channels extending through the hydrophilic foam layer;
a connection pad aligned with the one or more channels, the one or more channels providing fluid communication between the manifold layer and the connection pad, the connection pad coupleable to the tube to provide fluid communication between the pump and the manifold layer.

9. The negative pressure wound therapy system of claim 8, wherein the pump is manually powered.

10. The negative pressure wound therapy system of claim 8, the dressing further comprising a perforated film layer positioned under the manifold layer and allowing fluid to flow from the wound bed to the manifold layer.

11. The negative pressure wound therapy system of claim 8, wherein the hydrophilic foam layer is configured to absorb fluid from the manifold layer and the superabsorbent dots are configured to absorb fluid from the hydrophilic foam.

12. The negative pressure wound therapy system of claim 8, wherein the drape comprises a porous material that allows evaporation of fluid absorbed by the superabsorbent dots through the drape.

13. The negative pressure wound therapy system of claim 8, wherein the plurality of superabsorbent dots are separated from one another to facilitate deformation of the dressing.

14. The negative pressure wound therapy system of claim 8, the dressing further comprising:
a first fiber layer that binds the drape to the hydrophilic foam layer and secures the superabsorbent dots to the hydrophilic foam layer; and
a second fiber layer that binds the hydrophilic foam layer to the manifold layer.

15. The negative pressure wound therapy system of claim 8, the dressing further comprising a hydrophobic filter positioned between the one or more channels and the connection pad.

16. The negative pressure wound therapy system of claim 8, wherein the superabsorbent dots are maintained at substantially atmospheric pressure when the pump creates a negative pressure at the manifold layer.

17. A method of manufacturing a dressing, the method comprising:
printing a plurality of superabsorbent dots on a hydrophilic foam layer;
creating one or more channels through the hydrophilic foam layer;
coupling the hydrophilic foam layer to a drape, the superabsorbent dots positioned between the hydrophilic foam layer and the drape;
coupling a manifold layer to the hydrophilic foam layer in fluid communication with the one or more channels, the manifold layer substantially pneumatically isolated from the superabsorbent dots by the hydrophilic foam layer; and
coupling a connection pad to the drape in fluid communication with the one or more channels, the connection pad coupleable to a pump operable to create a negative pressure at the manifold layer.

18. The method of claim 17, wherein printing the plurality of superabsorbent dots on the hydrophilic foam layer comprises distributing a superabsorbent polymer in a pattern on the hydrophilic foam layer, the pattern comprising unconnected dots.

19. The method of claim 17, further comprising coupling a perforated film layer to the manifold layer, the perforated film layer coupleable to a wound bed and configured to allow fluid to flow from the wound bed to the manifold layer.

20. The method of claim 17, wherein the hydrophilic foam layer is configured to absorb fluid from the manifold layer and the superabsorbent dots are configured to absorb fluid from the hydrophilic foam layer.

21. The method of claim 17, wherein the drape comprises a porous material that allows evaporation of fluid absorbed by the superabsorbent dots through the drape.

22. The method of claim 17, wherein coupling the hydrophilic foam layer to the drape comprises binding the hydrophilic foam layer to the drape with a fusible fiber layer positioned between the drape and the hydrophilic foam layer, the fusible fiber layer securing the superabsorbent dots to the hydrophilic foam layer.

23. The method of claim 17, wherein coupling the manifold layer to the hydrophilic foam layer comprises fusing a fusible fiber layer between the hydrophilic foam layer and the manifold layer.

24. The method of claim 17, further comprising positioning a hydrophobic filter between the one or more channels and the connection pad.
